(19) United States
(12) Patent Application Publication
(10) Pub. No.: US 2007/0066911 A1
(43) Pub. Date: Mar. 22, 2007

(54) Integrated electrophysiology lab

(76) Inventor: Klaus Klingenbeck-Regn, Nurnberg (DE)

Correspondence Address: BRINKS HOFER GILSON & LIONE, P.O. BOX 10395, CHICAGO, IL, 60610, US

(21) Appl. No.: 11/231,675

(22) Filed: Sep. 21, 2005

(51) Int. Cl. A61B 5/04

(52) U.S. Cl. 600/523

(57) ABSTRACT

A system and method enhance the workflow of an electrophysiologist. A workstation may be interconnected with a number of medical devices associated with an EP lab. A user may enter patient registration data using the workstation that is subsequently sent to the medical devices. The medical devices may each perform a different electrophysiology related function and generate electrophysiology related data. The medical devices may send the data generated to the workstation. The workstation may perform operations on the data received to provide integrated results. The integrated results may include updating previously stored patient data and images or combining the data received to provide more detailed information and/or enhanced images. The integrated results may include displaying images and data from a number of medical devices on a display screen simultaneously. The workstation may generate a unified patient report based upon the data received from the medical devices.

### BACKGROUND

The present embodiments relate generally to medical devices. In particular, the present embodiments relate to medical devices associated with an electrophysiology lab.

An electrophysiology (EP) study of the heart is an analysis of the electrical conduction system (normal or abnormal) of the heart. The EP study is designed to find out the cause of heart rhythm disturbances and determine appropriate treatment. The test employs cardiac catheters and computers to generate electrocardiogram (EKG) tracings and electrical measurements taken from within the heart chambers.

An EP study may be performed solely for diagnostic purposes. An EP study also may be performed to pinpoint the exact location of electrical signals (cardiac mapping) in conjunction with a therapeutic procedure, such as catheter ablation. An EP study is performed in a special electrophysiology (EP) lab under controlled clinical circumstances by cardiologists and nurses who specialize in electrophysiology.

Conventional EP labs include a number of medical devices which each perform an electrophysiology related function. For example, the medical devices may be used for imaging, monitoring, and ablation purposes. Typical electrophysiology related medical devices are stand alone units each having dedicated keyboards, monitors, user interfaces, and other components. The configuration and volume of the medical devices associated with an EP lab may encumber an electrophysiology related workflow. Additionally, the EP lab and surrounding area may become overly crowded.

### BRIEF SUMMARY

By way of introduction, the embodiments described below include methods, processes, apparatuses, instructions, or systems for integrating medical devices to enhance electrophysiology workflow. A work station interconnects with a plurality of medical devices which each have electrophysiology related functionality. The workstation may integrate data received from the medical devices to present integrated results, including enhanced medical images. The workstation may permit the reduction of the number of stand alone medical devices and/or accompanying components within the EP lab.

In a first aspect, a data processing system enhances an electrophysiology workflow. The system includes a processing unit that generates integrated data from data received from a plurality of medical devices which each perform an electrophysiology related function. The system includes a display screen that displays integrated results generated from the integrated data.

In a second aspect, a data processing system includes a processing unit that receives data from a plurality of medical devices that perform different electrophysiology related functions. The system also includes a display screen that displays images generated from the data received which correspond to the different electrophysiology related functions.

In a third aspect, a method includes receiving data from a plurality of medical devices at a workstation associated with an electrophysiology lab. Each of the medical devices is operable for an electrophysiology related function. The method also includes generating integrated results using the data received.

In a fourth aspect, a computer-readable medium having instructions executable on a computer and stored thereon is described. The instructions include accepting data sets from a plurality of medical devices, the data sets containing information related to different electrophysiology related functions. The instructions also include integrating data using the data sets.

The present invention is defined by the following claims. Nothing in this section should be taken as a limitation on those claims. Further aspects and advantages of the invention are discussed below in conjunction with the preferred embodiments and may be later claimed independently or in combination.

### BRIEF DESCRIPTION OF THE DRAWINGS

The present embodiments will become more fully understood from the detailed description given herein below and the accompanying drawings which are given by way of illustration only, and are not limitative of the present invention, and wherein:

FIG. 1 is a block diagram of a conventional EP lab;

FIG. 2 is a block diagram of an exemplary integrated EP lab;

FIG. 3 is an exemplary method for enhancing an electrophysiology workflow;

FIG. 4 is a block diagram of an exemplary data processing system;

FIG. 5 is an exemplary user interface for enhancing an electrophysiology workflow; and

FIG. 6 is another exemplary user interface for enhancing an electrophysiology workflow.

### DETAILED DESCRIPTION OF THE DRAWINGS AND PRESENTLY PREFERRED EMBODIMENTS

A system and method provide an integrated electrophysiology (EP) lab. The integrated EP lab may enhance the workflow of medical personnel. The integrated EP lab integrates two or more medical devices to enhance the workflow within the EP lab. The integrated EP lab may be accomplished via either software or hardware, or a combination of software and hardware. The integrated EP lab may provide an integrated flow of medical data. The integrated flow of medical data may permit the single registration of patient data in the EP lab and/or allow the fusion of different types of EP related medical data.

I. Conventional EP Lab

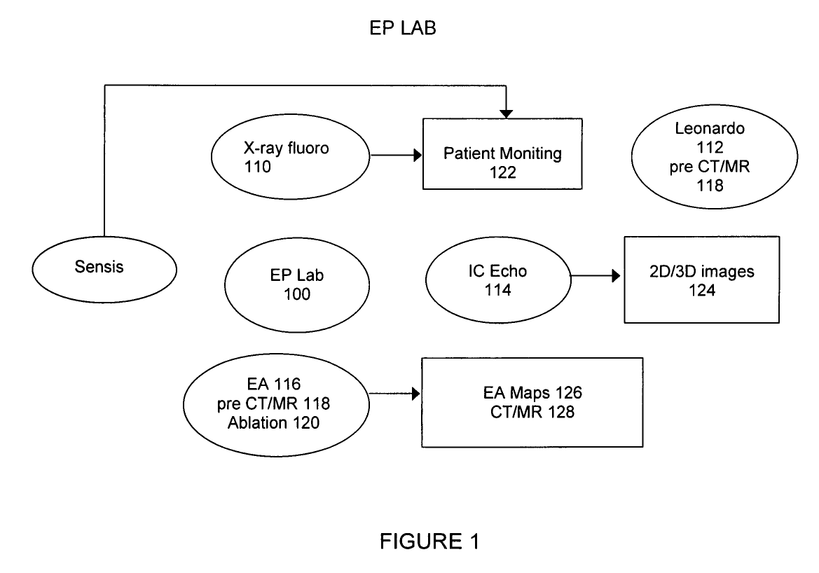

FIG. 1 illustrates a conventional EP lab 100. A conventional EP lab 100 may include several stand alone medical devices. As shown in FIG. 1, the medical devices may be dedicated to x-ray fluoroscopy 110, patient monitoring 112, intra-cardiac echo 114, electro-anatomical mapping 116, pre-interventional images 118, and/or other electrophysiology related functions. The medical devices also may include one or more generators that drive ablation catheters 120.

Conventional medical devices may each have their own keyboards, monitors, user interfaces, and other associated components. The example of FIG. 1 illustrates that a number of typical medical devices may each have a dedicated display screen for displaying information related to a specific electrophysiology related function. The conventional EP lab may have display screens directed toward displaying patient monitoring information 122, two or three dimensional medical images 124, electro-anatomical mapping 126 or computed tomography/magnetic resonance images 128, and/or other information.

However, the medical devices and accompanying display screens may be physically located throughout an EP lab. Medical personnel may have to routinely move about the EP lab to a different medical device and/or display screen to view patient images and data associated with a different type of electrophysiology related function. As a result, medical personnel may be prohibited from viewing all of the display screens and/or different types of electrophysiology related information conveniently from one location and/or simultaneously.

The stand alone medical devices which each provide separate and distinct functionality may encumber an electrophysiology workflow in other manners as well. As shown in FIG. 1, in a conventional EP lab, the medical devices are not directly interconnected with one another. Typically, there is no single network, data link, or data flow by which all of the medical devices may share data. As a result, the stand alone medical devices may hinder patient registration and may not permit the fusion, i.e., integration, of image, mapping, or other electrophysiology related data generated from multiple sources.

The conventional medical devices may require that a patient's demographic data be entered separately at each medical device, such as by a dedicated keyboard or other input device. The volume of medical devices and accompanying components, such as keyboards and display screens, may create an overly crowded working environment within the EP lab. Additionally, each of the medical devices may employ a different user interface utilizing distinct user operations.

II. Integrated EP Lab

The integrated EP lab may use a workstation to interconnect medical devices which each perform a different electrophysiology related function. The medical devices may be interconnected with the workstation via high speed data lines, wireless transmission, modem, computer network, common database, bus, or other means of transmission. Each of the medical devices may generate electrophysiology related data and send that data to the workstation. For example, the workstation may receive data, traces, images, and other information transmitted or generated by the medical devices. Alternatively, the workstation polls or requests the data. The workstation may integrate the data to provide an integrated data flow and integrated results. The integrated results may include updating patient data or combining data, traces, images and other information received to generate enhanced and more detailed information.

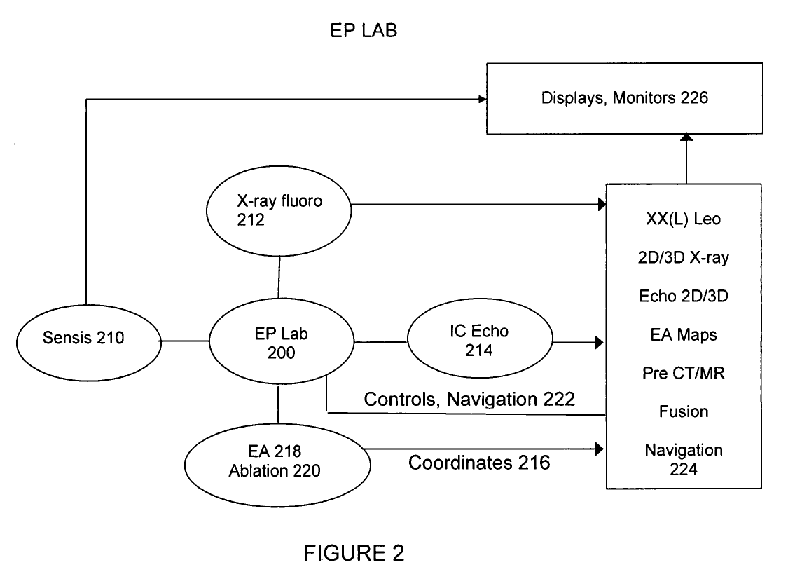

FIG. 2 is a block diagram of an exemplary integrated EP lab 200 that enhances an electrophysiology workflow. The integrated EP lab 200 may include an AXIOM Sensis hemodynamic and electrophysiological recording system 210, an x-ray fluoroscopic machine 212, and an intra-cardiac (IC) echo device 214. The integrated EP lap may include medical devices that provide mapping data with coordinates 216, such as a cardiac electro-anatomical (EA) mapping device 218 and/or a catheter ablation related device 220. The integrated EP lab 200 may include medical devices that provide control and navigation functionality 222. The integrated EP lab may include additional, fewer, or alternate medical devices.

The integrated EP lab 200 may include a data link that integrates the flow of data from one or more of the medical devices. As shown in FIG. 2, the integrated EP lab 200 may include a workstation 224 that may receive and integrate data from the medical devices. The workstation 224 may perform operations related to navigation, image and data fusion, pre-interventional computed tomography and/or magnetic resonance, electro-anatomical mapping, two and/or three dimensional intra-cardiac echos, two and/or three dimensional x-ray images, Leonardo workstations, and/or other electrophysiology related functionality.

The workstation 224 may be equipped with software that performs pre-procedural, procedural, and/or post-procedural processing. For example, the workstation 224 may evaluate image and/or non-image data received from the medical devices. The workstation 224 may update pre-procedural data with data received from an interventional procedure. The workstation 224 may update the pre-procedural data in real time providing assistance and current information during an interventional procedure.

The workstation 224 may generate integrated results by performing various operations on the data received. The workstation 224 may combine data received from one or more medical devices to create enhanced images and data. The data received may be received during a single treatment session. The workstation 224 may combine two and/or three dimensional images and use appropriate filtering to create more detailed images. The workstation 224 may rotate the two and/or three dimensional internal images on the display screen 226 at the direction of a user. The work station 224 may display different types of endoscopy related information simultaneously. The workstation 224 may be operable to direct and control one or more of the different endoscopy related devices.

The workstation 224 may fuse real time ultrasound images, including two and/or three dimensional images, from an intra-cardiac echo with previously acquired electro-anatomical maps. The workstation 224 may register or otherwise associate EKG (electrocardiogram) traces from a patient monitoring system to x-ray images of the same patient.

The workstation 224 may receive two and/or three dimensional pre-interventional CT (computed tomography) and/or MR (magnetic resonance) images. The workstation 224 may receive the pre-interventional images from the medical devices, a memory unit, an intranet, a radiology network of a medical facility, or other network. The workstation 224 may subsequently store the pre-interventional images, such as in a local memory.

The workstation 224 may integrate two and/or three dimensional data sets with electro-anatomical maps to add morphology, i.e., form and structure, to images reconstructed using the data sets. For example, intra-cardiac echo images may be fused with either CT and/or MR images to provide a real time update of the CT and/or MR morphology during an intervention.

The workstation 224 may receive two and/or three dimensional coordinates from a mapping device or system. All or some of the two and/or three dimensional data stored within or accessible by the workstation 224 may be used to support navigation related medical devices. For instance, the coordinate information may be used to control and steer navigation related medical devices in the EP lab. The navigation devices may include magnetic navigation devices, such as devices manufactured by Stereotaxis, and/or electromechanical navigation devices, such as devices manufactured by Hansen.

The workstation 224 may display the integrated results on one or more display screen(s) 226. The workstation 224 may use a single user interface on the display screens 226 such that data and images generated from different types of electrophysiology devices may be conveniently accessed and displayed using the same user operations or input devices. Providing access to images and data associated with a plurality of different electrophysiology related functions via a single interface may alleviate the need for medical personnel to learn the operation of a number of distinct user interfaces and their corresponding intricacies and nuances.

The workstation 224 may send data to each of the medical devices. As a result, the workstation 224 may facilitate sharing information between the medical devices. The data sent from the workstation 224 to the medical devices may have originated from another interconnected device. The data sent from the workstation 224 to the medical devices may be in an unaltered state or may include data modified and/or integrated by the workstation 224, such as data reformatted as appropriate for each medical device.

The capability of the workstation 224 to send data to and receive data from each of the medical devices may eliminate the need for medical personnel to manually enter that data at each of the individual medical devices. For example, a new patient may be registered a single time at the workstation 224 or another medical device. Subsequently, the workstation 224 may inform all of the interconnected medical devices of the patient's registration data, as well as other patient related data. As a result, a patient's registration data may only need to be entered once during a treatment session.

In one embodiment, the workstation 224 is interconnected with a large display screen. The user may be provided with the option of choosing between a number of electrophysiology related data sets for display on the display screen, each data set originating from a different medical device. The display screen may permit the user to concentrate on a single display screen instead of constantly turning his or her head or moving about the EP lab to view different display screens, each displaying a different type of EP related information.

The workstation 224 may provide the functionality of two or more conventional electrophysiology related medical devices. Alternatively, the workstation 224 may permit the reduction in the number of components associated with each stand alone medical device. For instance, the workstation 224 may permit the elimination of numerous display screens, keyboards, printers, monitors, input devices, output devices, accompanying electrical cords, and other components dedicated to individual medical devices. The reduction in the number of medical devices and/or accompanying components in, as well as in the vicinity of, the EP lab may alleviate a crowded work environment.

The workstation 224 may have a memory unit large enough to store all of the data sets received from the medical devices in a single database. The data sets may be visually represented on the display screen in various formats and sizes. The user interface may present information regarding each data set available for display, such as by an icon or bookmark. Alternatively, the workstation 224 accesses a medical records database or memories of the medical devices.

All of the data for a particular patient may be stored in a single folder. Each folder may include one or more sub-folders. Folder or sub-folder icons or other visual representations may indicate the individual medical device of origin for data associated with a folder or sub-folder, respectively. Alternatively, each folder or sub-folder may be represented by an alphanumeric or graphical mark indicating from where the data set originated. For example, for a computed tomography related data set, a folder or sub-folder may be called “CT” or be graphically represented by a computed tomography related symbol. Unified patient reports may be conveniently generated that include information from multiple medical devices.

III. Exemplary Embodiments

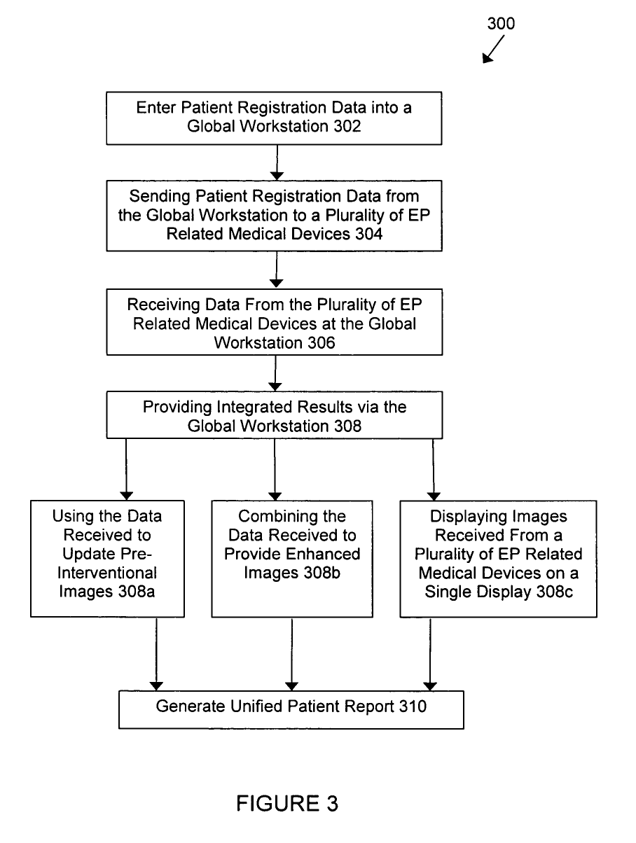

FIG. 3 is an exemplary method for enhancing an electrophysiology workflow 300. The method 300 may include entering patient registration data into a global workstation 302, sending the patient registration data from the global workstation to a plurality of EP related medical devices 304, receiving data from the plurality of EP related medical devices at the global workstation 306, providing integrated results via the global workstation 308, and generating a unified patient report 310. The method may include additional, fewer, or alternate actions.

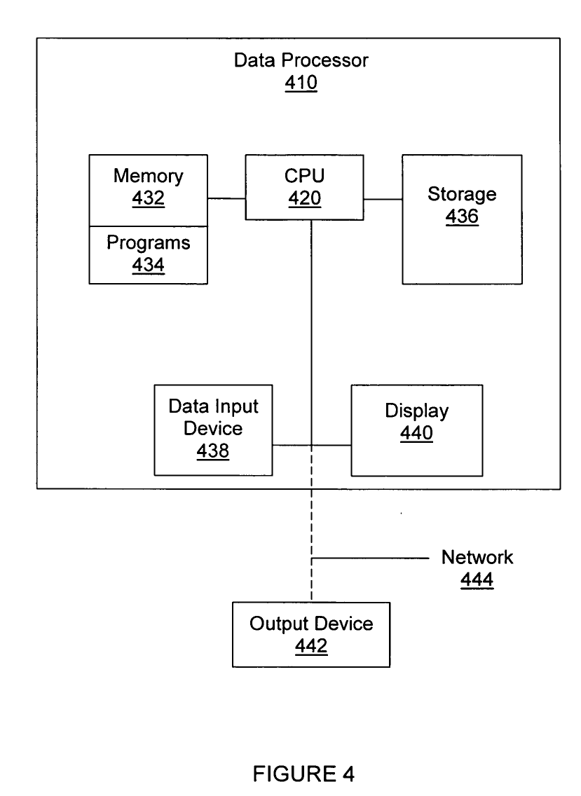

The method for enhancing an electrophysiology workflow 300 may be facilitated by a global workstation or data processing system interconnected with a number of medical devices. FIG. 4 is a block diagram of an exemplary data processor 410 configured or adapted to provide functionality for enhancing an electrophysiology workflow. The data processor 410 may include a central processing unit (CPU) 420, a memory 432, a storage device 436, a data input device 438, and a display 440. The data processor 410 also may have an external output device 442, which may be a display, a monitor, a printer and/or a communications port. The data processor 410 may be a personal computer, work station, server, one of the medical devices, or other system. The data processor 410 may be interconnected to a network 444, such as an intranet, the Internet, or an intranet connected to the Internet. The data processor 410 may be interconnected to another location via the network 444 either by data lines or by wireless communication. The data processor 410 is provided for descriptive purposes and is not intended to limit the scope of the present system. The data processor may have additional, fewer, or alternate components.

The network 444 may interconnect the data processor 410 with a plurality of medical devices. Alternatively, the medical devices may each be directly, indirectly, or wirelessly interconnected with the data processor 410. Each of the medical devices may perform electrophysiology related functionality. The medical devices interconnected with the data processor 410 may include different types of electrophysiology related devices, including x-ray fluoroscopic, patient monitoring, intra-cardiac echo, electro-anatomical mapping, ablation catheter, computed tomography, ultrasound, and magnetic resonance devices. The data processor 410 may be interconnected with additional, fewer, or alternate types of electrophysiology related devices. The data processor 410 may direct that the data received be stored on or read from machine-readable medium, including secondary storage devices such as hard disks, floppy disks, CD-ROMS, and DVDs; electromagnetic signals; or other forms of machine readable medium, either currently known or later developed.

A program 434 may reside on the memory 432 and include one or more sequences of executable code or coded instructions that are executed by the CPU 420. The program 434 may be loaded into the memory 432 from the storage device 436. The CPU 420 may execute one or more sequences of instructions of the program 434 to process data. The program 434 may provide workflow assistance and functionality as discussed herein.

The method 300 may include entering patient registration data into a global workstation 302. As shown in FIG. 4, the program 434 may permit a user to enter patient registration data into the global workstation or data processor 410 via the data input device 438, the network 444, or another input device. After which, the patient registration data may be stored in the memory 432, the storage device 436, or other storage unit.

The method 300 may include sending the patient registration data from the global workstation to a plurality of EP related medical devices 304. For instance, the program 434 may direct the data processor 410 to send the patient registration data, as well as other electrophysiology related data, to the medical devices interconnected with the data processor 410. Additionally, the patient registration data or other data processed by the data processor 410 may be provided as an output to the display 440, the external output device 442, the network 444, and/or stored in a database.

The method 300 may include receiving data from the plurality of EP related medical devices at the global workstation 306. The data may be received via the network 444 or other network with which the medical devices are interconnected. The data processor 410 may receive and store the medical data received in the memory 432, the storage device 436, or other storage unit. The program 434 may direct that the data received be stored on or read from machine-readable medium, including secondary storage devices such as hard disks, floppy disks, CD-ROMS, and DVDs; electromagnetic signals; or other forms of machine readable medium, either currently known or later developed.

The method 300 may provide integrated results via the global workstation 308. The program 434 may direct the data processor 410 to perform one or more operations on the data received to generate integrated results. The data processor 410 may display the integrated results on the display 440, output device 442, or other output means.

Providing integrated results via the global workstation 308 may include using the data received to update pre-interventional images 308a, combining the data received to provide enhanced images 308b, and/or displaying images received from a plurality of EP related medical devices on a single display 308c. The data processor 410 may generate the integrated results by combining data received from a number of medical devices, by updating data stored in the memory 432, storage 436, or other storage unit, or by displaying data received from two or more the medical devices simultaneously on the display 440, output device 442, or other output means. Additional, fewer, or alternate integrated results may be provided.

The method 300 may include using the data received to update pre-interventional images or other previously stored data 308a. The data processor 410 may receive and store pre-interventional related data, such as data generated from an x-ray, CT, MR, ultrasound, or other non-invasive medical device. Subsequently, during an interventional procedural, the data processor 410 may receive internal images and other data. The internal images and other data may be integrated with the non-invasive data to provide enhanced or adjacent images and other data in real time for use during and/or after the procedure.

The method 300 may include combining the data received to provide enhanced images and other information 308b. The data processor 410 may receive and combine two and/or three dimensional images from medical devices providing different functionality as discussed herein. For example, the data processor 410 may use a three dimensional image associated with one type of EP related functionality to update or enhance a two dimensional image associated with another type of EP related functionality. For instance, CT or MR images may be used to enhance x-ray or electro-anatomical images, or vice versa. Other combinations may be used.

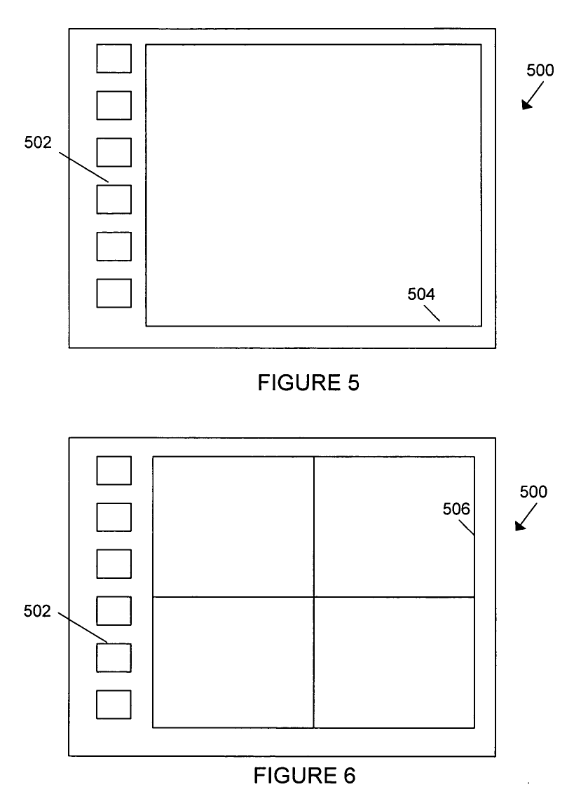

The method 300 may include displaying images received from a plurality of EP related medical devices on a single display 308c. FIG. 5 illustrates an exemplary user interface for presenting the integrated results. As shown, the user interface 500 may include one or more icons 502 and a primary window 504. The user interface 500 may include additional, fewer, or alternate components.

Each icon 502 may be associated with a different EP related function. Alternatively, each icon 502 may be associated with a different integration of multiple EP related functions. For example, an icon 502 may be associated with a data set that is the result of integrating one or more types of EP related data with CT, MR, electro-anatomical mapping, intra-cardiac echo, or other EP related data types as discussed herein. Other combinations may be used.

The primary window 504 may display the integrated results discussed herein, including images and data associated with one or more EP related functions. An operation performed on an icon 502, such as by a mouse, touch screen, or other input device, may result in the images and/or data displayed in the window 504 being changed to those associated with that icon 502. As a result, the images and data from a plurality of EP related medical devices may be displayed via a single display screen that uses a single user interface.

FIG. 6 illustrates another exemplary user interface for presenting the integrated results. The user interface 500 may include a number of icons 502 and windows 506. The user interface 500 may include additional, fewer, or alternate components.

Each icon 502 may be associated with a different EP related function or a different integration of multiple EP related functions. Each window 506 may display the integrated results discussed herein, including images and data associated with one or more EP related functions. An operation performed on an icon 502 may result in the images and/or data displayed in all or some of the windows 506 being changed to those associated with that icon 502.

For instance, all four windows 506 may be changed to present information related to an EP function associated with the icon 502 in response to an operation performed on that icon 502. Alternatively, a single window 506 may be changed to present information related to an EP function associated with that icon 502, while the remaining windows 506 may display information related to different EP functions. As a result, the images and data from different EP related medical devices may be displayed via a single display screen that uses a single user interface simultaneously.

The exemplary user interfaces of FIGS. 5 and 6 may provide functionality for rotating and/or translating along one or more axes of the two or three dimensional images received from the medical devices discussed herein. The exemplary user interfaces may permit the images to be superimposed over one another to emphasize additional features, changes to the images, or other differences.

In one embodiment, the windows of the exemplary user interfaces may be directed toward displaying (1) pre-interventional images, (2) images obtained during an interventional procedural, (3) updated or integrated images that integrate the pre-interventional images with the images obtained during the interventional procedure, and (4) images that highlight or only show the areas in the integrated images that are updated, i.e., the difference in the pre-interventional and interventional images. By emphasizing the differences in the pre-interventional and interventional images, or between other images, in a single window, medical personnel may more easily identify areas that have more detail in one set of images, have changed over time, or are of interest. The user interface may display all of the pre-interventional, interventional, and integrated images, as well as the images emphasizing differences between two or more sets of images, individually or simultaneously.

The method 300 may include generating a unified patient report 310. The data processor 410 may use the data received from more than one of the medical devices to generate a detailed patient report. The detailed patient report may be based upon analysis of x-ray, CT, MR, electro-anatomical, intra-cardiac, catheter ablation, pre-procedural, procedural, post-procedural, and other electrophysiology related images and data. The patient report that combines numerous types of information and analysis into a single report may provide efficiencies in an electrophysiology workflow. For instance, one or more reports dedicated to a single type of EP related medical device may be eliminated.

While the invention has been described above by reference to various embodiments, it should be understood that many changes and modifications can be made without departing from the scope of the invention. The description and illustrations are by way of example only. Many more embodiments and implementations are possible within the scope of this invention and will be apparent to those of ordinary skill in the art.

It is intended in the appended claims to cover all such changes and modifications which fall within the true spirit and scope of the invention. Therefore, the invention is not limited to the specific details, representative embodiments, and illustrated examples in this description. Accordingly, the invention is not to be restricted except in light as necessitated by the accompanying claims and their equivalents.

I claim:

1. A data processing system for enhancing an electrophysiology workflow, the system comprising:
a processing unit operable to generate integrated data from data received from a plurality of medical devices, each of the plurality of medical devices being operable to perform an electrophysiology related function; and
a display screen operable to display integrated results generated from the integrated data.

2. The system of claim 1, wherein the processing unit is operable to generate the integrated data by updating previously stored data with the data received from the plurality of medical devices.

3. The system of claim 1, wherein the processing unit is operable to generate the integrated data by combining the data received from the plurality of medical devices.

4. The system of claim 1, wherein the display screen displays electrophysiology related images generated from data received from two or more of the plurality of medical devices simultaneously.

5. The system of claim 1, wherein the processing unit is operable to accept patient registration information and subsequently share the patient registration information with the plurality of medical devices such that the patient registration information need only be entered once during a treatment session.

6. The system of claim 1, wherein the processing unit is operable to generate a unified report that is based upon the data received from the plurality of medical devices.

7. The system of claim 1, comprising a user interface operable to switch between different types of electrophysiology images being displayed on the display screen.

8. The system of claim 1, wherein the plurality of medical devices include at least two different types of electrophysiology devices, the different types of electrophysiology devices include x-ray fluoroscopic, intra-cardiac echo, electro-anatomical mapping, ablation catheter, computed tomography, or magnetic resonance devices.

9. A data processing system for enhancing an electrophysiology workflow, the system comprising:
a processing unit operable to receive data from a plurality of medical devices, each of the plurality of medical devices being operable to perform a different electrophysiology related function; and
a display screen in an electrophysiology lab, the display screen operable to display images generated from the data received, the images displayed on the display screen correspond to a plurality of different electrophysiology related functions.

10. The system of claim 9, wherein the plurality of different electrophysiology related functions include at least two of x-ray fluoroscopic, intra-cardiac echo, electro-anatomical mapping, ablation catheter, computed tomography, or magnetic resonance related functions.

11. The system of claim 9, wherein the images are generated by integrating the data received from two or more of the plurality of medical devices.

12. The system of claim 9, wherein the images are generated by using the data received to update pre-interventional images.

13. The system of claim 9, wherein the processing unit is operable to generate a unified patient report based upon the data received from the plurality of medical devices.

14. A method for enhancing an electrophysiology workflow, the method comprising:
receiving data from a plurality of medical devices at a workstation associated with an electrophysiology lab, each of the plurality of medical devices performs an electrophysiology related function; and
generating integrated results using the data received.

15. The method of claim 14, wherein the integrated results are displayed on a display associated with the workstation.

16. The method of claim 14, wherein generating integrated results comprises updating pre-interventional images with data received from at least one of the plurality of medical devices.

17. The method of claim 14, wherein generating integrated results comprises combining the data received from the plurality of medical devices to provide enhanced images, the enhanced images being displayed on a display screen associated with the workstation.

18. The method of claim 14, wherein generating integrated results comprises displaying images generated from different electrophysiology related functions on a single display screen associated with the workstation.

19. The method of claim 14, comprising:
entering patient registration data into the workstation; and
sharing the patient registration data with the plurality of medical devices such that the patient registration data only needs to be entered once.

20. The method of claim 14, comprising generating a single report for a patient based upon the data received from the plurality of medical devices.

21. The method of claim 14, wherein the data received from the plurality of medical devices is received during a treatment session.

22. A computer-readable medium having instructions executable on a computer stored thereon, the instructions comprising:
accepting data sets from a plurality of medical devices, the data sets containing information related to different electrophysiology related functions; and
generating an integrated flow of data using the data sets.

23. The computer-readable medium of claim 22, comprising displaying integrated results on a single display screen, the integrated results include a plurality of images which each correspond to different electrophysiology related functions.

24. The computer-readable medium of claim 22, comprising generating integrated results by combining the data sets to generate an enhanced two or three dimensional electrophysiology related image.

25. The computer-readable medium of claim 22, comprising generating integrated results by updating patient data stored in a memory unit.
